(19) United States
(12) Patent Application Publication
(10) Pub. No.: US 2008/0242935 A1
(43) Pub. Date: Oct. 2, 2008

(54) Endoscope

(76) Inventor: Takahiro Inoue, Tokyo-to (JP)

Correspondence Address: KENYON & KENYON LLP, ONE BROADWAY, NEW YORK, NY, 10004, US

(21) Appl. No.: 12/074,995

(22) Filed: Mar. 7, 2008

(30) Foreign Application Priority Data
Mar. 29, 2007 (JP) 2007-086219

(51) Int. Cl. A61B 1/06

(52) U.S. Cl. 600/176

(57) ABSTRACT

An endoscope incorporates an objective optical system and an image sensor at its distal end and has light guides around the objective optical system. In this case, when a direction parallel to the major side of an imaging region of the image sensor is taken as a lateral direction, exit end faces of the light guides are arranged only in the lateral direction of the objective optical system, a top cover is placed opposite to the exit end faces, and an illumination light diffusing portion is provided at the position of the top cover opposite to the exit end faces. Whereby, the endoscope in which the small-diameter design of the distal end is attained and the loss of emergent light is minimized can be provided.

This application claims benefits of Japanese Application No. 2007-86219 filed in Japan on Mar. 29, 2007, the contents of which are incorporated herein by reference.

### BACKGROUND OF THE INVENTION

1. Field of the Invention

This invention relates to an endoscope, and in particular, to a small-diameter endoscope.

2. Description of Related Art

Generally, in an endoscope, its body is constructed with a slender insertion tube section and a manipulation section provided at a proximal end of the insertion tube section. The insertion tube section includes an objective optical system for forming an image of an observation object, an image sensor for converting the image into an electric signal, and an illumination means for supplying illumination light to the observation object, provided inside the insertion tube section at a distal end.

There are a number of various endoscopes in which channels into which treatment tools are inserted and channels for water feed and suction are provided inside the insertion tube sections. However, the small-diameter endoscope exclusively used for observation generally has only the objective optical system, the image sensor, and the illumination means.

In recent years, high-density pixel designs of imaging devices have been developed, and even in the small-diameter endoscopes, the wide use of display regions such as laterally elongated monitors with aspect ratios of 5:4, 4:3, and 16:9 is being increased.

In order to obtain such a laterally elongated display region, the image sensor is, of course, laterally elongated, and an observation area on an object surface also becomes a laterally elongated region. However, in a central rotationally symmetric illumination means using a perfectly circular illumination lens and a light guide fiber whose tip is molded into a cylindrical shape, which formerly has been used, an illumination area on the object surface is also perfectly circular and the loss of illumination light is caused. As a technique of preventing this loss of illumination light, it is conceivable that the illumination area is also laterally elongated like the observation area.

In order to obtain such a laterally elongated illumination area, it is known that, as shown in FIG. 1, light guides 13 and illumination lenses 14 are arranged on both sides of an objective optical system 12, and the light guides 13 and the illumination lenses 14 are decentered (Japanese Patent Kokai No. Hei 11-326786).

### SUMMARY OF THE INVENTION

The endoscope of the present invention has features described below.

(1) The endoscope incorporates an objective optical system and an image sensor at its distal end and has light guides around the objective optical system. In this case, when a direction parallel to the major side of an imaging region of the image sensor is taken as a lateral direction, exit end faces of the light guides are arranged only in the lateral direction of the objective optical system, a top cover is provided opposite to the exit end faces, and an illumination light diffusing portion is provided at the position of the top cover opposite to the exit end faces.

(2) In item (1), the illumination light diffusing portion includes a plurality of concentric minute grooves, with the optical axis of the objective optical system as a center.

(3) In item (1), the illumination light diffusing portion has an annular surface.

(4) In item (2), each of the minute grooves has a width of 0.1 mm or less.

(5) In item (2), the minute grooves are superposed on the annular surface.

(6) In items (1)-(5), the illumination light diffusing portion has negative refracting power.

(7) In item (6), when a direction perpendicular to the lateral direction is taken as a vertical direction, the illumination light diffusing portion has larger negative refracting power in the lateral direction than in the vertical direction.

(8) In items (1)-(7), the top cover is shaped to cover at least a part of the side surface of the distal end.

(9) In items (1)-(7), the whole of the top cover is made of transparent resin.

(10) In items (1)-(7), the top cover is constructed so that at least the illumination light diffusing portion is made of transparent resin and remaining portions are made of opaque resin, the transparent resin and the opaque resin being integrally molded by a dichromatic molding process.

(11) In items (1)-(7), the top cover is constructed so that at least the illumination light diffusing portion is made of optical glass and remaining portions are made of opaque resin, the optical glass and the opaque resin being integrally molded.

According to the present invention, the endoscope in which the small-diameter design of the distal end is attained and the loss of emergent light is minimized can be provided.

These and other features and advantages of the present invention will become apparent from the following detailed description of the preferred embodiments when taken in conjunction with the accompanying drawings.

### BRIEF DESCRIPTION OF THE DRAWINGS

FIG. 1 is a sectional view showing schematically the optical arrangement of a conventional endoscope;

FIG. 2 is a view showing the contour of the endoscope of Embodiment 1 in the present invention;

FIG. 3 is a horizontal sectional view showing of the endoscope of Embodiment 1;

FIG. 4 is a view showing a modified example of the top cover of the endoscope of Embodiment 1;

FIG. 5 is a partially cutaway perspective view showing the structure of the endoscope of Embodiment 1;

FIG. 6A is a front view showing the distal end of the endoscope of Embodiment 1;

FIG. 6B is a sectional view taken along line A-A in FIG. 6A;

FIG. 6C is a sectional view taken along line B-B in FIG. 6A;

FIG. 7 is a partially cutaway perspective view showing the structure of the endoscope of Embodiment 2 of the present invention;

FIG. 8 is a sectional view showing the illumination light diffusing portion of the endoscope of Embodiment 3 of the present invention;

FIG. 9 is a sectional view showing a modified example of the illumination light diffusing portion of the endoscope of Embodiment 3; and

FIG. 10 is a view showing the contour of the endoscope of Embodiment 4 of the present invention.

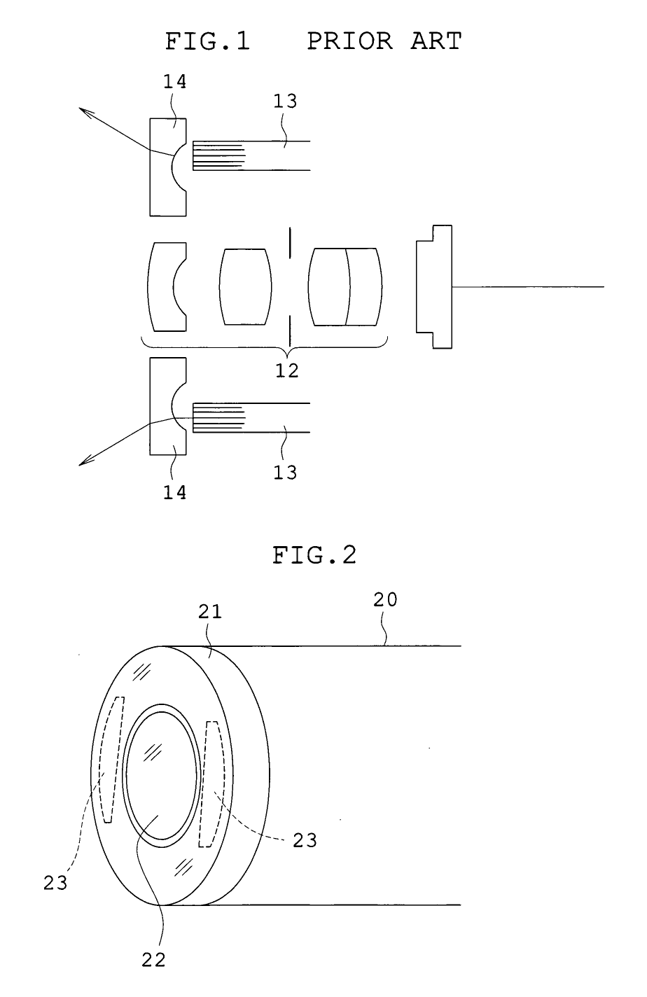

### DETAILED DESCRIPTION OF THE PREFERRED EMBODIMENTS

In accordance with the drawings, the embodiments of the present invention will be explained below.

### Embodiment 1

FIG. 2 shows the contour of the endoscope of this embodiment. An objective optical system 22 is incorporated in a distal end 20 of the endoscope so that it is exposed to the exterior, and light guides 23 are arranged around the objective optical system 22. A top cover 21 which is opposite to the exit end faces of the light guides 23, covers a part of the side surface of the distal end 20, and is made of transparent resin as a whole is fixed by cementation.

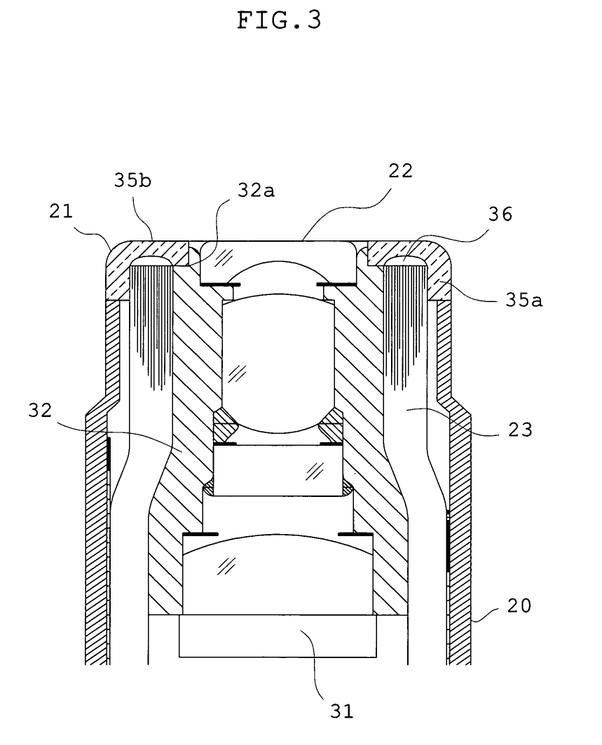

FIG. 3 shows the horizontal section of the distal end 20. At the distal end 20, an image sensor 31 with a laterally elongated imaging region is placed at the imaging position of the objective optical system 22 in a lens barrel 32.

The top cover 21 includes a peripheral barrel portion 35a covering the side surface of the distal end 20 and an annular portion 35b configured integrally with the peripheral barrel portion 35a and is provided with an illumination light diffusing portion 36 including an annular surface at the position of the annular portion 35b opposite to the exit end faces of the light guides 23.

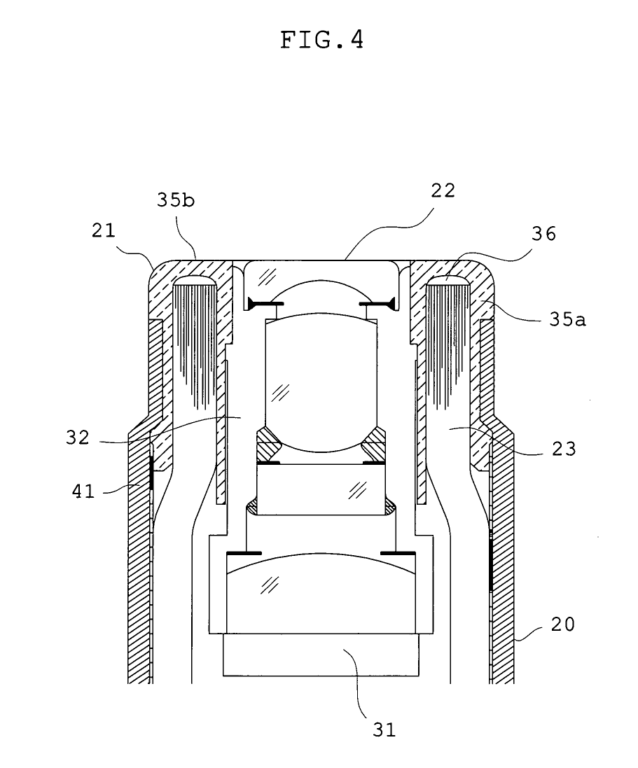

Also, the top cover 21 may be constructed so that the lens barrel 32 is configured with a step portion 32a and is fitted into, and cemented to, the top cover 21. However, in order to further improve a watertight property, as shown in FIG. 4, it is rather desirable that the peripheral barrel portion 35a is sufficiently elongated in the longitudinal direction of the endoscope and is cemented and fixed to both surfaces of a soft tube 41 covering the side surface of the distal end 20 and the lens barrel 32.

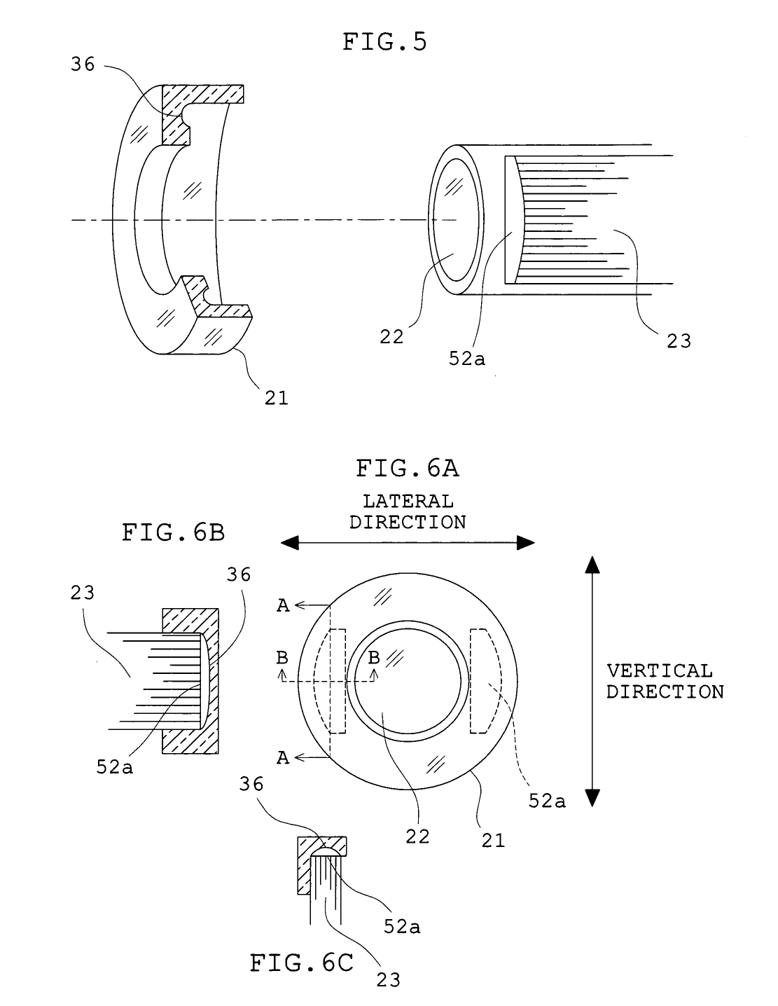

The structure of the endoscope of Embodiment 1 is shown in FIG. 5. The illumination light diffusing portion 36 provided at the position of the top cover 21 opposite to exit end faces 52a of the light guides 23 includes a rotationally symmetric annular surface, with the center of the top cover 21 as an axis of symmetry, and is capable of diffusing illumination light emerging from the exit end faces 52a because it functions as a concave lens with negative refracting power. Also, when a direction parallel to the major side of the imaging region of the image sensor is taken as a lateral direction, the light guides 23 are arranged only in the lateral direction of the objective optical system 22 so that they are not arranged in a vertical direction perpendicular to the lateral direction.

By the arrangement mentioned above, the illumination light emerging from the exit end faces 52a of the light guides 23 is diffused, uniformly distributed light is obtained, and a favorable observation image can be formed.

In addition, as shown in FIGS. 6A-6C, the illumination light diffusing portion 36 is such that the radius of curvature is smaller in the lateral direction than in the vertical direction and thus the negative refracting power is greater in the lateral direction. Consequently, the illumination light is strongly diffused in the lateral direction so that a laterally elongated illumination area is obtained, and the observation image with uniform brightness is formed without wasting the amount of emergent light. Further, since the tips of the light guides 23, unlike conventional cylindrical shapes, are configured to be vertically slender, the layout of the distal end is obtained with little loss and a small-diameter design of the endoscope can be easily attained.

Moreover, since the illumination light diffusing portion 36 is constructed with a simple rotationally symmetric body, its fabrication is easy and satisfactory performance is obtained by only the operation that after the positional adjustment of the light guides 23 is made with respect to the objective optical system 22 on assembly, they are fitted into the top cover 21. Hence, a special positional adjustment is not necessary and an assembly property is excellent.

### Embodiment 2

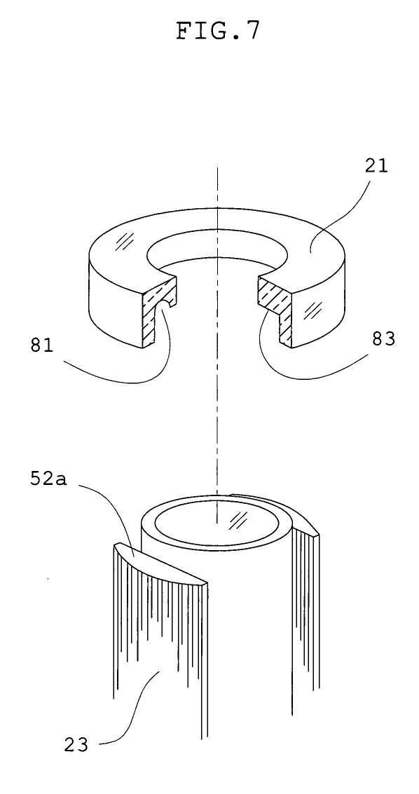

The structure of the endoscope of this embodiment is shown in FIG. 7. Parts not shown in this figure are the same as in Embodiment 1 and their explanation is omitted. In Embodiment 2, portions of the top cover 21 that are not opposite to the exit end faces 52a of the light guides 23 are provided as plane portions 83. It is only necessary that illumination light diffusing portions 81 are provided at positions opposite to the exit end faces of the light guides. The plane portions 83 are provided like Embodiment 2 and thereby the property of cementation between the top cover 21 and the lens barrel can be improved.

### Embodiment 3

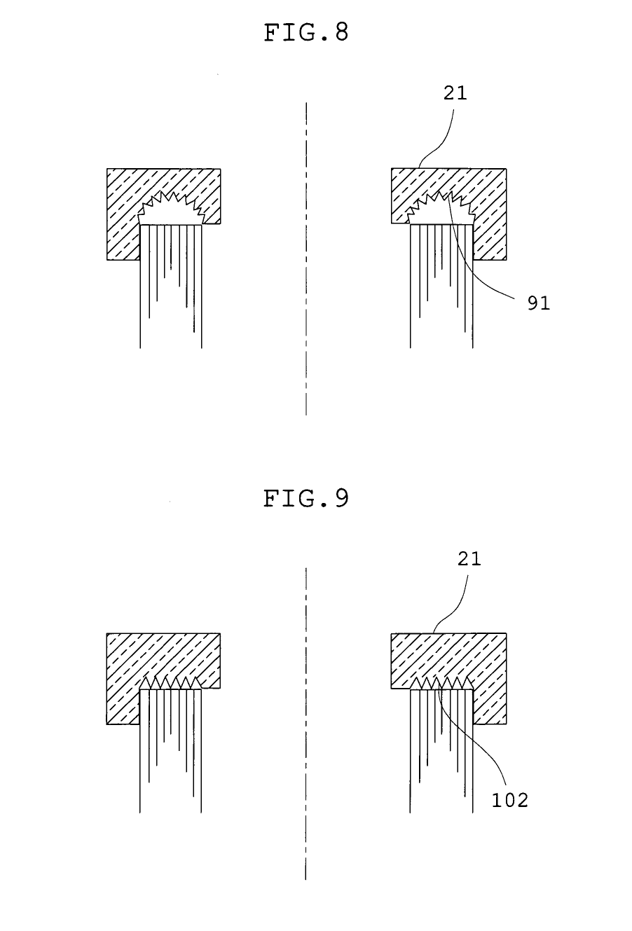

FIG. 8 illustrates the section of the illumination light diffusing portion of the endoscope of this embodiment. In Embodiment 3, a plurality of concentric minute grooves 91 are superposed on the annular surface, with the center of the top cover 21 as an axis of symmetry, and are configured as the illumination light diffusing portion. It is desirable that the width of each groove is 0.1 mm or less. By the function of the plurality of minute grooves 91, the negative refracting power is increased in the lateral direction, but it is little changed in the vertical direction, and thus a more laterally elongated illumination area is obtained. As illustrated in FIG. 9, even when only a plurality of concentric minute grooves 102 are configured as the illumination light diffusing portion, the same effect is brought about.

### Embodiment 4

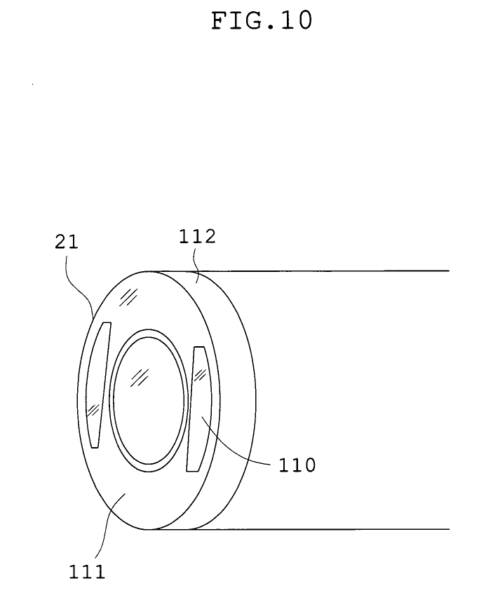

The contour of the endoscope of this embodiment is shown in FIG. 10. In Embodiment 4, of the top cover 21, the illumination light diffusing portion is made of transparent resin 110, and the annular portion and the peripheral barrel portion excluding the illumination light diffusing portion are made of opaque resins 111 and 112, respectively, so that both portions are integrally molded by a dichromatic molding process. In Embodiment 4, since internal structure of the endoscope at the distal end does not meet the eyes of a doctor and a patient, this top cover 21 excels in grade, as compared with the case where the whole of the top cover 21 is molded out of transparent resin.

The top cover 21 may be molded in such a way that an optical glass member independently molded into the illumination light diffusing portion is embedded in opaque resin. By using the optical glass member with a high refractive index as the illumination light diffusing portion, stronger negative refracting power is attained and the observation image that has uniform brightness at all corners of a visual field is obtained even in the endoscope with a wide field angle.

In the top cover, like Embodiment 4, any transparent material of which the illumination light diffusing portion is made brings about a satisfactory effect.

Although individual embodiments have been explained as mentioned above, the present invention is not limited to the embodiments and various modifications and changes can be made without departing from the scope and spirit of this invention.

What is claimed is:

1. An endoscope which incorporates an objective optical system and an image sensor at a distal end thereof and has light guides around the objective optical system,
wherein when a direction parallel to a major side of an imaging region of the image sensor is taken as a lateral direction, exit end faces of the light guides are arranged only in the lateral direction of the objective optical system, a top cover is placed opposite to the exit end faces, and an illumination light diffusing portion is provided at a position of the top cover opposite to the exit end faces.

2. An endoscope according to claim 1, wherein the illumination light diffusing portion includes a plurality of concentric minute grooves, with an optical axis of the objective optical system as a center.

3. An endoscope according to claim 1, wherein the illumination light diffusing portion has an annular surface.

4. An endoscope according to claim 2, wherein each of the minute grooves has a width of 0.1 mm or less.

5. An endoscope according to claim 2, wherein the minute grooves are superposed on an annular surface.

6. An endoscope according to claim 1, wherein the illumination light diffusing portion has negative refracting power.

7. An endoscope according to claim 6, wherein when a direction perpendicular to the lateral direction is taken as a vertical direction, the illumination light diffusing portion has larger negative refracting power in the lateral direction than in the vertical direction.

8. An endoscope according to claim 1, wherein the top cover is shaped to cover at least a part of a side surface of the distal end.

9. An endoscope according to claim 1, wherein a whole of the top cover is made of transparent resin.

10. An endoscope according to claim 1, wherein the top cover is constructed so that at least the illumination light diffusing portion is made of transparent resin and remaining portions are made of opaque resin, the transparent resin and the opaque resin being integrally molded by a dichromatic molding process.

11. An endoscope according to claim 1, wherein the top cover is constructed so that at least the illumination light diffusing portion is made of optical glass and remaining portions are made of opaque resin, the optical glass and the opaque resin being integrally molded.
